(19) United States
(12) Patent Application Publication
(10) Pub. No.: US 2022/0218966 A1
(43) Pub. Date: Jul. 14, 2022

(54) TRIGEMINAL NEURALGIC BALLOON COMPRESSION KIT

(71) Applicant: Orhan BARLAS, Istanbul (TR)

(72) Inventor: Orhan BARLAS, Istanbul (TR)

(21) Appl. No.: 17/632,161

(22) PCT Filed: Jan. 3, 2020
(86) PCT No.: PCT/TR2020/050004

§ 371 Date: Feb. 1, 2022

(30) Foreign Application Priority Data
Oct. 16, 2019 (TR) 2019/15950

(51) Int. Cl. A61M 29/02; A61M 25/09; A61M 25/06

(52) U.S. Cl. CPC A61M 29/02; A61M 25/09; A61M 2210/005; A61M 2210/0687; A61M 25/0662

(57) ABSTRACT

The present invention relates to a medical kit and method of administration for trigeminal balloon compression used in the treatment of trigeminal neuralgia. The kit reaches the damaged area percutaneously and penetrates the balloon most effectively to the damaged spot. The present invention is a trigeminal neuralgic balloon compression kit which can overcome the aforementioned disadvantages; it is characterized by being a product kit and application method which has a simple structure and can easily and effectively convey the balloon to the damaged area.

### TECHNOLOGICAL FIELD

This invention concerns a medical kit and its application method for trigeminal balloon compression used in treatment of trigeminal neuralgia.

### STATE OF THE ART

Trigeminal Neuralgia is a neurological disease that inflicts severe pain to a part of the face. It is a condition that is mostly unilateral, brief, characterized by flashing pains, instantaneous, limited by the diffusion range of one or two branches of the trigeminal nerve; that is, around the eye, in the region of the cheekbone and the cheeks, or in the region of the lower jaw. While the pain may be triggered by insignificant stimuli such as touching a particular spot on the face, cold weather, chewing, speaking, swallowing, washing the face, shaving, or smoking; it may also occur frequently spontaneously.

This disease occurs in the nerve that transmits sensation in the face, the trigeminal nerve, which is the fifth cranial nerve. Incidence of the disease is about 4.3 in 100.000. The disease is more common in women and in the elderly. The unexpected pain effects life quality, is mentally challenging and sometimes and may even drive patients to suicide.

Initial treatment of trigeminal neuralgia is carried out with drugs (carbamazepine). Surgical methods are used when the drug is not effective or loses its effect. Surgery is performed on the trigeminal nerve. These methods can be divided into three groups: open surgery, percutaneous procedures, and radiosurgery. Today, the most known and most effective operations are “microvascular decompression” that are performed through craniotomies, and trigeminal radiofrequency rhizotomy (RF) and trigeminal balloon compression performed percutaneously.

The microvascular decompression (MVD) method eliminates vascular compression of the nerve root as it exits the brainstem relieving the neuralgia. This procedure is an open, microsurgical major brain surgery performed between the deepest and most delicate structures and vessels of the brain. Therefore, there is a serious morbidity and mortality rate of around 5%. As with other brain surgeries, there are no incisions in the skin, so these are closed surgeries.

In the trigeminal radiofrequency rhizotomy (RF) method, a lesion is made by heating the nerve root behind the trigeminal ganglion with a controlled radiofrequency current in the skull base. This procedure requires the use of special electrodes and radiofrequency generators which are expensive and not generally available. The procedure is usually performed under mild sedation local anesthesia as it requires patient cooperation, and may be quite painful. Postoperatively partial loss of facial sensation occurs in most patients, and when this is severe, as it occurs in 2% of patients, it may result in severe neuropathies (anesthesia dolorosa) or loss of vision.

The percutaneous balloon compression is performed under general anesthesia as patient participation is not required for a successful outcome. A needle is percutaneously advanced to the “foramen ovale” at the base of the skull using landmarks and under radioscopic guidance. Once the the needle enters the skull through the foramen to reach the trigeminal nerve ganglion, a balloon catheter (Fogarty 4F) is delivered through it and inflated with 0.75 ml of radio opaque liquid for 3 minutes. During this period, the nerve root behind the ganglion is compressed between the balloon and bone, and its pain-transmitting fibers, which are presumably more vulnerable than the fibers that transmit other sensory modalities, become unable to transmit pain. Other functions of the nerve remain intact. With this method, treatment is performed with a success rate of 75%-85%.

Percutaneous balloon compression has a relatively high success rate, very low complication rate and does not need sophisticated, high cost equipment. Yet it has not become popular. The reason for this is the difficulty of inserting a large needle into the oval foramen.

Consequently, there is a need for a medical equipment which can overcome the above-mentioned disadvantages, has a simple structure and can be produced easily and cost-effectively.

### DESCRIPTION OF THE INVENTION

The present invention is a trigeminal neuralgic balloon compression kit which can overcome the aforementioned disadvantages; it is characterized by being a product kit and application method which has a simple structure and can easily and effectively convey the balloon to the damaged area.

Prior to the present invention, there was difficulty in accessing the trigeminal region.

These disadvantages are eliminated with the invention kit. It facilitates accession of the trigeminal ganglion region percutaneously and the procedure can be performed in a short time.

Another feature of the invention is that it comprises guide wires of different calibers. In this way, the appropriate caliber for the patient can be determined and applied to the correct point effectively.

A further feature of the invention is that the sheath and then the balloon can be easily inserted by means of the guide wire and thereby perform its function.

### EXPLANATION OF THE FIGURES

The invention be described with reference to the accompanying drawings from now on. Thus, the features of the invention will be more clearly understood and appreciated. However, this is not intended to limit the invention to certain embodiments. On the contrary, it is intended to cover all alternatives, modifications, and equivalents that may be included within the scope of the invention as defined by the appended claims.

FIG. 1 Sheath Frontal View,

FIG. 2 Sheath Side view,

FIG. 3 Guide Wire (caliber 1.8),

FIG. 4 Fixing Notch Close-up View,

FIG. 5 Guide Wire (caliber 1.6),

FIG. 6 Guide Wire (caliber 1.4),

FIG. 7 Guide Wire (caliber 1.1),

FIG. 8 Sheath and Guide Wire Mount View,

FIG. 9 Sheath and Guide Wire Edge Close-up

The figure to assist in the understanding of the present invention is numbered as indicated in the attached drawing and is given below along with their names.

### EXPLANATION OF REFERENCES

1. Sheath head
2. Guide Wire (caliber 1.8)
3. Guide Wire (caliber 1.6)
4. Guide Wire (caliber 1.4)
5. Guide Wire (caliber 1.1)
6. Sheath and Guide Wire Edge Close-up

### DESCRIPTION OF THE INVENTION

Trigeminal Neuralgia Balloon Compression kit, which is the subject of the invention, consists of five parts. The first part of the equipment consists of four guide wires, which are respectively 1.8 mm, 1.6 mm, 1.4 mm and 1.1 mm in diameter. Marking notches are provided on the guide wires. This notch indicates how far the sheath will be advanced over the wire.

The sheath needle passes safely over this guide to provide a path for the placement of the balloon catheter. The outer caliber of the sheath is 2.2 mm and the inner caliber is 2 mm.

### DETAILED EXPLANATION OF THE INVENTION

In percutaneous balloon compression of trigeminal neuralgia, neuralgia is treated by application of compression to the trigeminal nerve root posterior to the ganglion with a temporarily inflated balloon.

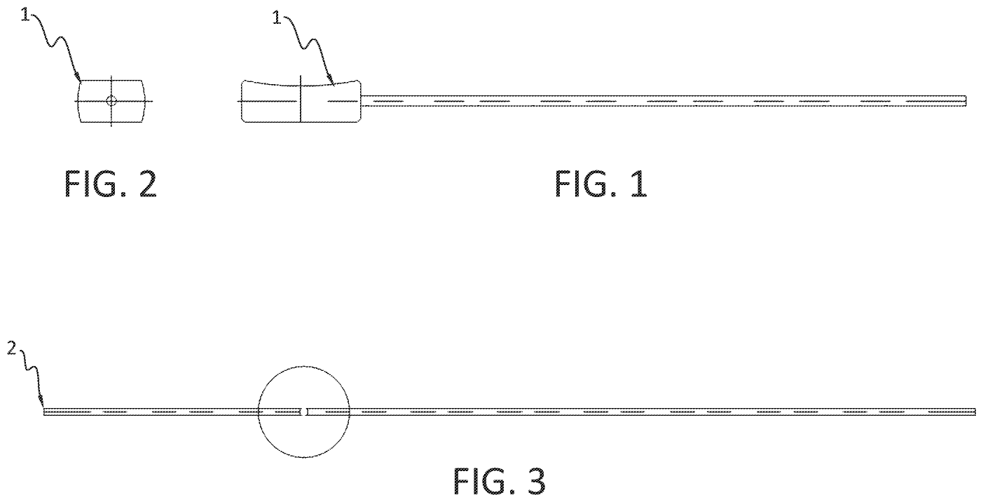

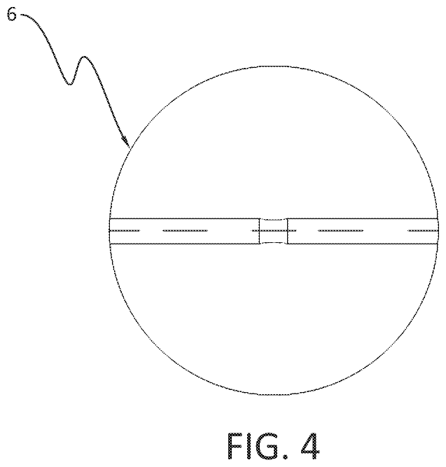

The guide wire is inserted into the foramen oval percutaneously. The surgeon feels that the foramen has been entered and confirms this radiographically. The wire is advanced to the “clivus” line under radioscopic control. The sheath (FIG. 1) is advanced over this wire to the marker notch in the wire being inserted into the foramen (FIG. 3, 5, 6, 7). Radioscopy confirms again that the sheath has engaged the foramen. At this stage, the guidewire is withdrawn, the balloon is inserted through the catheter sheath and inflated with 0.75 ml radio-opaque material. Inflation of the balloon is checked by radioscopy. After waiting for three minutes, the liquid in the flask is withdrawn and the system is ejected.

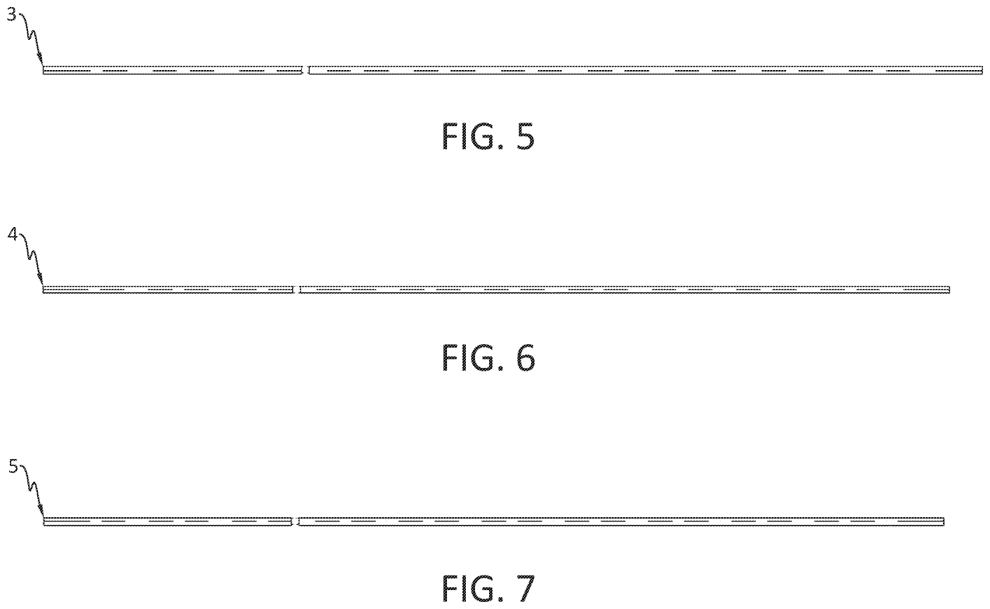

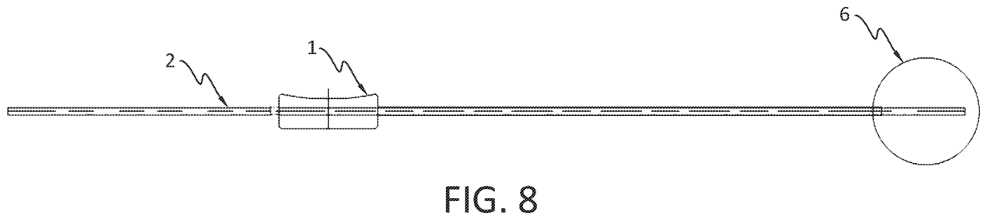

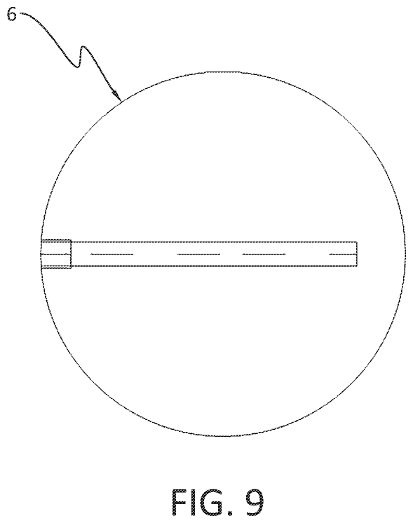

The sheath (FIG. 1) consists of two parts as a cap and a guide body. The head part is used for manual fixation and the body part (1) is used percutaneously to penetrate the trigeminal region. The guide wire (FIGS. 3, 5, 6, 7) passes through the body part (1). The edge of the sheath is not sharp.

The four guide wires in the kit (FIGS. 3, 5, 6, 7) have a caliber of 1.8 mm, 1.6 mm, 1.4 mm, 1.1 mm. The ends of the wires are blunt. The caliber of the foramen ovale may vary from patient to patient. Therefore, when it is not possible to treat some patients with large caliber wires, small caliber wires are required.

There is an indication notch on the wire. This notch, which can be felt by touch, is also colored for visual identification. The location of the notch is related to the length of the sheath and the length of the balloon, and is 17 mmm longer than the length of the sheath in the present kit.

In the present kit, the length of the sheath is 156 mm, the length of the wire is 240 mm, and the marking point on the wire is 173 mm (156+17). However, the length of the sheath can be reduced to 80 mm if desired, but the mark on the wire should equal the length of the inflatable tip of the balloon catheter, usually between 15 and 20 mm.

1.-4. (canceled)

5. A catheter system for delivering a compression balloon through the foramen ovale of the skull of a patient, comprising:
a plurality of rigid guide wires having different diameters, and
a sheath having an inner diameter large enough to slidably receive the guide wire of the plurality of guide wires having the largest diameter,
wherein a system operator
selects one of the plurality of guide wires having a diameter small enough to insert through the foramen oval of the patient,
inserts the selected guide wire through the foramen ovale of the skull of the patient, and
slides the sheath over the selected guide wire to deliver a compression balloon.

6. The system of claim 5, wherein the diameters of the plurality of guide wires are 1.1 mm, 1.4 mm, 1.6 mm, and 1.8 mm, respectively.

7. The system of claim 5, wherein the sheath is formed from one of stainless steel, chrome plated steel, aluminum, and a plastic composition.

8. The system of claim 5, wherein each of the plurality of guide wires is formed from one of stainless steel, chrome plated steel, aluminum, and a plastic composition.

9. A method for delivering a compression balloon through the foramen ovale of the skull of a patient to relieve trigeminal neuralgia using a catheter kit that includes a plurality of rigid guide wires having different diameters, and a sheath having an inner diameter large enough to slidably receive the guide wire of the plurality of guide wires having the largest diameter, comprising the steps of:
selecting one of the plurality of guide wires having a diameter small enough to insert through the foramen oval of the patient,
inserting the selected guide wire through the foramen ovale of the skull of the patient under the guidance of radioscopy until the back of the trigeminal nerve ganglion is reached, and
sliding the sheath over the selected guide wire to deliver a balloon catheter against the trigeminal nerve ganglion.
